(19) United States
(12) Patent Application Publication
(10) Pub. No.: US 2026/0176427 A1
(43) Pub. Date: Jun. 25, 2026

(54) Procedure for the purification of biodegradable thermoplastic polymeric particles for medical and/or pharmaceutical use

(71) Applicant: Laboratorios Farmaceuticos ROVI S.A., MADRID (ES)

(72) Inventors: IBON GUTIERRO ADURIZ, MADRID (ES); Maria GARCIA AMO, MADRID (ES); ELENA Cebadera Miranda, MADRID (ES)

(73) Assignee: Laboratorios Farmaceuticos ROVI S.A., MADRID (ES)

(21) Appl. No.: 19/540,239

(22) Filed: Feb. 13, 2026

Related U.S. Application Data
(62) Division of application No. 18/054,356, filed on Nov. 10, 2022.
Application No. 18/054,356 is a continuation-in-part of application No. PCT/ES2020/070306, filed on May 12, 2020.

(51) Int. Cl. C08J 3/12; A61K 9/50; B29C 71/02

(52) U.S. Cl. CPC C08J 3/12; A61K 9/5031; A61K 9/5089; C08J 7/08; C08J 2367/04

(57) ABSTRACT

Procedure for the purification of biodegradable thermoplastic polymer particles for medical and/or pharmaceutical use without the use of organic solvents, as well as the particles obtained themselves, and the use of polymeric particles obtained by this procedure to manufacture parenteral administered medicinal products and/or implantable medical devices.

### CROSS-REFERENCE TO RELATED APPLICATIONS

The present application claims the benefit of and is divisional of application Ser. No. 18/054,356 filed Nov. 10, 2022, which is a continuation-in-part of international application No. PCT/ES2020/070306, filed May 12, 2020, the entire disclosure of which is hereby incorporated by reference.

### FIELD OF THE INVENTION

The present invention falls within the field of purification of polymeric particles obtained from biodegradable thermoplastic polymers in order that they can be used in the medical, pharmaceutical and/or health fields. In particular, the process and particles of the present invention have the technical advantage of being obtained in a non-pyrogenic environment and low microbial load. More particularly, it is a process for the purification of polymeric particles in the absence of solvents, which preferably consist of polymer or copolymers of lactic acid, glycolic acid, and/or caprolactone. The present invention also relates to the particles themselves, to the use of these particles in the health field, such as in biodegradable medical devices (catheters, sutures, staples, surgical clips, implants) as well as in the formulation of implantable drugs (intramuscular or subcutaneous).

### BACKGROUND OF THE INVENTION

Synthetic biodegradable thermoplastic polymers have been used for years for medical applications, and their use is becoming more and more widespread. Most of these biodegradable polymers are solid thermoplastic materials based on polymers and copolymers of lactic and glycolic acids and caprolactone. The administration of these biodegradable devices requires in some cases the practice of a surgical incision to place the solid material at the incision site. In other variants, the biodegradable polymer is dissolved in an organic solvent, and the liquid formulation is administered using for example a syringe and a needle, resulting in a solid implant at the site of deposit of the formulation by precipitation of the polymer when it comes into contact with body fluids. One of the applications of these formulations consisting of synthetic biodegradable thermoplastic polymers is the addition of an active component to the formulation, giving rise in this case to polymer-active implants from where the release of the active ingredient is controlled by the biodegradation of the biodegradable polymer. Other applications are the formation of catheters, sutures, staples, or biodegradable surgical clips as support material in medical applications.

The biodegradable plastic polymers (PLGA, PLA, etc.) are generally found in parenteral pharmaceutical preparations in the form of concentrated polymer solutions in organic solvents, and since, on the one hand, they have a strong tendency to form intermolecular esters (eg, dimeric and polymeric lactic acid) and on the other, are strongly hygroscopic products, the rate of degradation of the polymer in these formulations is higher than that of the dry polymer, which hinders purification on an industrial scale, which is a very complicated and laborious procedure. In addition, to facilitate the formulation of the ultimate drug or medical device, polymer particles of a small size or size distribution are generally required to be procured for the final drug or medical device. These polymer processing steps to reduce particle size, for example by grinding or crushing, contribute to the contamination of the product obtained, reducing its purity by up to 20% compared to that of the initial product.

As these are polymers for sanitary applications, to guarantee quality, the European and American guidelines set very strict mandatory requirements (such as the control of pressure, temperature and humidity level) that, logically, are applicable to pharmaceutical products [PDA J. Pharm. Sci and Tech. 2015, 69 123 139, (draft) PDA J. Pharm. Sci and Tech. 2018]. Additionally, in the case of parenteral formulations of difficult visual inspection, as is the case of a parenteral formulation consisting of an active ingredient and a polymer, the guides require an accessory test to verify that the product is essentially free of unwanted or extrinsic particles. This additional test-specified in the chapters of the American guidelines published by the American Pharmacopoeia [USP 1790 Visual Inspection of Injections and USP 790 Visual Particles in Injections]-provides for a visual inspection of a sample representative of the batch size, after dissolving them in a suitable solvent. Under visual inspection, no visible particles should be observed in the selected samples. According to the literature published to date, particles larger than 100-150 μm are considered visible.

The importance of detecting these visible particles in time before being inserted or implanted inside the human body is of paramount importance, since parenteral administration of inert particles can lead to four pathogenic mechanisms: (i) infections and local or systemic inflammations caused by the presence of microorganisms or endotoxins; (ii) inflammatory response caused by the particle or derivatives thereof, which can cause tissue damage; (iii) allergic or anaphylactic reactions; and (iv) tissue damage caused by blood capillary occlusion (PDA J. Pharm. Sci and Tech. 2015, 69 123 139, (draft) PDA J. Pharm. Sci and Tech. 2018). For this reason, for the release to the market of this type of medical or pharmaceutical products with an adequate quality assured, it is necessary to develop a procedure that allows the removal of such inert or solvent-insoluble particles with a size greater than 100-150 μm.

The most common sources of unwanted particles in the manufacture of a parenteral medicine or in an implantable medical device are the procedure for obtaining them (the medicine or device), since the use of raw materials, packaging materials, equipment, the work environment and the personnel involved in the entire manufacturing process may have a significant impact upon product quality and efforts to reduce unwanted particles may be costly.

With regard to the state of the art, it is necessary to mention that there are processes for preparation and polymerization of well-known thermoplastic polymers and that, by way of illustration, they are constituted by the following general stages: (i) polymerization reaction, (ii) elimination of residual monomers, (iii) extrusion, (iv) cooling and molding, and (v) final conditioning. The final molding of the polymer can take many forms: pellets, catheters, spheres, particles, etc.; however, none of the state-of-the-art processes provides a sterile polymer with low content of unwanted particles.

Polymerization reactions to obtain thermoplastic polymers (both homopolymers and copolymers) for parenteral use such as lactic acid polymers (L,D,DL), glycolic polymers and/or ε-caprolactone such as poly polymer (L-lactic), poly (D-lactic), poly (DL-lactic), polyglycolic, poly (ε-caprolactone), copolymers of L-lactic/S-lactic, L-lactic/DL-lactic acids, L-lactic/DL-lactic, L-lactic/glycolic, DL-lactic/glycolic or L-lactic/ε-caprolactone, among others, can be carried out under a diverse variety of temperature, time and pressure conditions. These polymerization reactions are preferably carried out in the absence of oxygen.

In state-of-the-art processes, polymerization is generally carried out by adding the starting monomers to the reactor, closing it, and pressurizing it, generally using inert gas, such as nitrogen, oxygen or hydrogen, among others. Then the reactor begins to heat with a slow temperature ramp, between 0.1° C./min and 5.0° C./min, from room temperature. Once the monomers have partially melted, agitation begins, and stirring continues until the reactor has reached a temperature of at least 5° C. above the melting temperature (Tm) of the monomers. At this time, the agitation is stopped, and the reactor is depressurized. The catalyst is then incorporated into the reactor, the reactor is shut down and pressurized with inert gas. The agitation is restarted and maintained for long enough to achieve a homogeneous mixture of all the components. Once this homogeneous mixture is obtained, the agitation is stopped, and the reactor is depressurized. The initiating agent is incorporated into the reactor, the reactor is closed and pressurized with inert gas. The agitation is restarted and maintained until the reactor mixture has reached a certain viscosity. It is at that time, when the agitation stops, and both the temperature and the positive pressure of the inert gas are maintained for as long as it takes for the polymerization to complete, typically between 9 and 35 hours. Once the polymerization is completed, the residual monomers are removed. For this, the temperature is maintained, and a slight agitation and vacuum is applied to the molten mixture, for the volatilization of the residual monomers. Finally, the polymer is extruded from the reactor. To do this, the temperature is maintained, and the agitation is stopped, pressurized with inert gas and a discharge valve is opened so that the molten polymer flows from the reactor. At the exit of the reactor, the polymeric fluid solidifies by cooling with water until the polymer is solid and reserved for later use, either in the form of particles, granules, pellets, etc. The polymeric particles obtained by the polymerization process described above are marketed by pharmaceutical manufacturers; however, the present inventors have determined that, despite being sold as “pharmaceutical quality”, the prior art particles do not have the degree of purity necessary to be used in parenteral systems (drugs or medical devices), so the inventors have been forced to purify them by a dry (non-solvent-based) reprocessing method of the invention, in order to keep intact the physicochemical properties of the starting particles but without particles of compounds or substances foreign to the structure of the polymer of choice.

Japanese patent JP2012025968 to NIPPON CATALYTIC CHEM IND describes a pelletization process for obtaining a thermoplastic acrylic resin. A polymerization reaction provides the thermoplastic acrylic resin. After this, the resin is extruded using a temperature within the range of 220 to 300° C. Once the extrusion is carried out, the resin is filtered by means of a metal filter with a pore size of 10 μm to eliminate contaminating particles of a particle size of 20 μm. Once the resin is filtered, it is cooled by water, which is in a temperature range of 30 to 80° C. Finally, it is pelletized to a pellet of 4 mm in size. The process does not contemplate air cooling.

Chinese utility model CN206796507 to CHONGQING YOUHE NEW MAT CO LTD describes a plastic extruding device composed of two extruders, each with a filter, plus a cooling device connected to the second extruder. The device also consists of a pelletizer. The process does not contemplate air cooling and requires a double extrusion and filtering.

Patent No. U.S. Pat. No. 5,439,688 to DEBIO RECHERCHE PHARMACEUTIQUE S.A. describes a procedure for the preparation of a pharmaceutical composition in the form of microparticles. A polymer is mixed together with a pharmaceutical salt. The resulting mixture is compressed and heated (maximum temperature of 90° C.) before entering the extruder. In the extruder, the mixture remains at a temperature between 9° and 100° C., and after extrusion it is necessary to cool the mixture obtained by thermal transfer to a cooled sterile gas or air. Finally, the product is crushed by cryogenic crushing (0° C. or even −30° C.). The process does not remove particles greater than 100 μm in size, and the process requires filtration of the extruded polymer for removal of unwanted particles.

International Publication WO 2000035423 of AVENTIS PHARMACEUTICALS Inc. describes a method for producing a pharmaceutical composition based on a fusion extrusion method to form microparticles. The steps carried out to obtain the microparticles consist of mixing the polymer with a pharmaceutical salt to form a dry mixture, extruding this mixture until a homogeneous mixture is obtained in the form of a strand, cooling the mixture and pelletizing this strand, and pulverizing the pellets to form microparticles with sizes between 10 to 200 μm. This process employs spray or wet spraying to obtain the final formulation. Again, this process does not take into account the removal of impurities from the polymer sample because the final object is to ensure that the final formulation has the active ingredient in combination with the polymer in the form of a polymer matrix.

On the other hand, the international publication WO2015/028060 A1 of EVONIK INDUSTRIES AG describes a process for preparing an absorbable polyester in powder form obtained by a dissolution-precipitation process using organic solvents, with a specific density lower than that of the starting polymer. The patent describes a solvent purification process, where the physicochemical properties (such as the specific density of the purified product) vary from those of the starting polymer. In fact, the process describes a redensification step to increase the density of the resulting product, but the density value of the starting polymer is not reached in any case. The filtration step of the process is for removal of solvent from the particles.

Chinese patent application CN105111423 A of PENG CHUNHAI describes the manufacture of a water-based polyester by condensation, and it employs organic solvents in its process. To obtain the polymer, the steps of (a) esterification, (b) filtration, (c) condensation polymerization reaction, and (d) extrusion of the mixture are followed. Filtration is conducted prior to polymerization to eliminate monomers that are not of interest in the mixture of esters to be polymerized. Filtration is not conducted on the extruded polymer after its formation.

There are also several patent applications describing different types of filtration devices for extrusion processes of thermoplastic polymers.

International publication No. WO2017/163180 A1 of C M PRODUZIONE S.R.L. describes a device for the continuous filtering of molten plastic materials that come from the plastic waste recycling industry, wherein the plastic waste has a high presence of inorganic impurities. In this case, the filters are used to remove inorganic products from the extrusion mixture, mainly heavy metals. The process does not filter the already-formed particles.

International publication No. WO2001/47687 A2 of UNION CARBIDE CHEMICAL & PLASTICS TECHNOLOGY CORPORATION discloses a filtering device for molten polypropylene and ethylene propylene copolymers, in which the filter is intended to remove residues and aggregates from polymeric materials. This international patent application describes a filter and a method for filtering molten polymers, and the described method consists of several stages: polymer melting, melting the molten polymer, extrusion to shape it, and cooling. This international patent application presents the stages of filtration and extrusion but in the reverse order, since in the international application the extrusion is used to shape the copolymer obtained and the filtration is used to remove the monomers of the starting polymers that do not matter to be present in the final copolymer. This is because, what is pursued with filtration and subsequent extrusion, is separation of the large particles of agglomerate of the molten copolymer to later mix the filtered melt.

Chinese utility model CN202213190 of ANTEPU ENGINEERING PLASTICS SUZHOU CO LTD discloses a filtration device with a coupled vacuum pump for extrusion processes without interruptions due to filter blockage. The filter aims to remove residues from the melting process of highly polymeric materials. The process is intended for the recycling of non-biodegradable plastics.

Other art describes specific filters coupled to extruders, e.g. patent No. U.S. Pat. No. 8,202,423 (Pub. US20080314815 A1) of GNEUSS or patent No EP3088157 A2 of FIMIC S.r.l. Such filters, however, are unsuitable for use according to the present invention.

International publication No. WO2018069238 A1 of DR COLLIN GMBH describes a device and method for inspecting molten polymers made from plastic materials. The device consists of an extruder, a storage device, a pressure filter test device, and an electronic control device coupled to the extruder. To identify molten polymers, the process employs a code identification device, such as QR codes, or barcodes. In addition, it has an extruder, a storage device where the identification device is located, a pressure filter test device and an electronic control device coupled to the extruder.

The filters described in the aforementioned patents are standard quality metal filters for the manufacture of non-implantable and non-parenteral industrial copolymers. In addition, the filtration process of molten polymers described in these patents can be carried out in an environment with standard cleaning requirements. Moreover, to cool the polymer for solidification the melt is passed through a container with cold water. Cooling is not conducted with an inert gas stream.

None of the prior art processes concerning filtration of molten polymer control the temperature of the polymer with regard to its melting temperature in combination with the temperature of cooling of the polymer with regard to its glass transition temperature. As a result, none of the prior art processes are capable of providing purified polymer particles that have substantially the same physicochemical and/or rheological properties as the corresponding starting material of unpurified polymer particles.

Due to the various disadvantages inherent in the current state-of-the-art processes, there is a need for development of a process for the purification of biodegradable thermoplastic polymers, without the use of solvents and without altering the physicochemical properties of the polymer, for use in demanding sanitary applications, such as obtaining implantable medical devices or parenteral pharmaceutical formulations (intramuscular, subcutaneous, intravenous, etc.) that require a polymer purity level of at least 95%, preferably at least 97% and more preferably 99%, without fibers of a nature other than that of the polymer.

### SUMMARY OF THE INVENTION

The present inventors have developed an alternative process for eliminating extrinsic particles from, i.e. purifying, preformed particulate biodegradable thermoplastic polymers, wherein the physicochemical and/or rheological properties of the starting product and the purified product are substantially the same or substantially unaltered.

An aspect of the invention provides for the use of purified polymeric particles and/or microparticles in the manufacture of parenterally administered drug dosage forms and/or implantable medical devices.

The invention also provides purified biodegradable thermoplastic polymeric particles included within drug dosage forms and/or implantable medical devices. The invention also provides a method of treating a disease, disorder or condition comprising administration of the purified biodegradable thermoplastic polymeric particles, administration of medical devices comprising said polymeric particles, or administration of dosage forms comprising said polymeric particles. In preferred embodiments, the drug dosage forms are parenterally administered.

The invention also provides a method of manufacturing medical devices from the purified biodegradable thermoplastic particles and also provides the corresponding medical devices.

An aspect of the invention provides the particles that are a product of the process of the invention characterized in that the particles are sterile and suitable for parenteral formulations or for the manufacture of implantable medical devices.

The polymeric particles, e.g. purified biodegradable thermoplastic polymeric particles, of the invention are characterized by being essentially free of visible extrinsic particles, with a polymer purity level of at least 95%, preferably at least 97% and more preferably 99%, wherein the purified particles exhibit substantially the same physicochemical characteristics, e.g. the same inherent viscosity, the same bulk density, the same molding ability, etc.) as those of the starting unpurified polymeric particles, i.e. the particles prior to purification according to the process. In some embodiments, the purified particles exhibit a customized particle size distribution. Generally, the properties of the purified polymeric particles will depend on the properties of the starting unpurified polymeric particles. In some embodiments, the bulk density (p b) and the compacted density (pt, from “tap or tapped density”) of purified polymeric particles obtained are substantially the same as those of the corresponding unpurified preformed polymeric particles, i.e. the starting material.

In some embodiments, the purified polymeric particles are characterized by the absence of residual solvent. This is achievable, because the process of the invention employs dry purification, wherein no solvent is used in the purification process as described here. In some embodiments, the amount of residual solvent present in the purified polymeric particles is typically no more than 0.00%, or not more than 0.000%, or not more than 0 ppm, calculated as the total amount of solvent per total weight of biodegradable thermoplastic polymer. However, where the starting unpurified polymeric particles have been previously purified using some type of solvent, it is possible that the purified polymeric particles of the inventio might contain residual amounts of solvent(s). Preferably the starting polymer particles have not been previously purified, or have not been previously purified, with a method that uses solvent(s). In particular, in a process as described here, it is preferably characterized because the biodegradable thermoplastic polymer provided in step a) has not been previously purified or has not been previously purified with a method using solvents.

Generally, the purified polymeric particles are characterized by a residual solvent content (e.g. acetone) of less than 0.5%, in particular less than 0.3%, more particularly less than 0.1%, and more particularly less than 0.05%, even less than 0.01%, calculated as total amount of solvent by total weight of biodegradable thermoplastic polymer.

The invention also provides an alternative polymeric particle purification process for eliminating visible extrinsic particles of a size greater than 100-150 μm from a powder of polymeric particles. As used herein, “extrinsic particles” refers to those particles of a nature or composition different from that of the starting polymeric material, e.g., any amorphous or defined particle of polymeric origin, such as fibers, granules, pellets, agglomerates, aggregates (e.g. aggregates from the fusion of the polymer itself), among others that have a composition different than that of the preformed polymeric particles being purified. In particular, extrinsic particles can be organic, such as natural cellulose fibers, or inorganic such as metals. A pharmaceutical product or medical device made from said purified polymeric particles will thus not contain said visible extrinsic particles.

The purification process of the invention is a solvent-free purification process, wherein preformed powdered polymeric particles comprising extrinsic particles are purified, without the use of solvent, by melting said preformed particles and then extruding and filtering the molten polymer, and then cooling of the filtered polymer, and then comminuting of the cooled polymer to provide purified powdered polymeric particles free from visible extrinsic particles of a size greater than 100-150 μm or greater than 150 μm.

The process of the invention is performed under sterile conditions, e.g. non-pyrogenic conditions and low microbial load conditions. Moreover, the process of the invention results in elimination of all particles of size greater than 100-150 μm from the starting powdered polymeric particles.

The product of the solvent-free purification process of the invention provides purified powdered polymeric particles essentially free of visible extrinsic particles, with a polymer purity level of at least 95%, preferably at least 97% and more preferably 99%, wherein the purified particles exhibit substantially the same physicochemical characteristics of the starting product (i.e. the same inherent viscosity, the same bulk density, the same molding ability, etc.). The purified particles optionally exhibit a customized particle size distribution suitable for use in a parentally administered dosage form or medical device.

An aspect of the invention provides a solvent-free (dry) purification process for purifying a powder comprising polymeric particles, the method comprising:
a) providing a preformed particulate biodegradable thermoplastic polymer in a reactor and then melting the polymer;
b) extruding and filtering the molten polymer at least once through at least one filter having a pore size of about 5-300 μm, wherein the extruding and filtering is conducted at a temperature that is 4.5-5.5° C. (or not more than about 5.5° C.) above the melting temperature (Tm) of the selected polymer and with positive pressure greater than 0.1 bar, in the presence of an inert gas;
c) cooling the filtered polymer by means of a sterile gas stream to a temperature lower (less) than that of step b) until the polymer reaches a temperature of at least 4.5-5.5° C. below the glass transition temperature (Tg) of the polymer; and
d) comminuting the cooled polymer to obtain purified polymeric particles with a particle diameter ≥1 mm.

In some embodiments, the cooling stage is carried out by exposing the extruded polymer to a gas stream at a temperature below that of stage b) to ensure that the polymeric particles reach a temperature of at least 4.5-5.5° C. below the glass transition temperature (Tg), since at this temperature an optimal thermodynamic pseudotransition occurs in glassy materials (glasses, polymers, and other amorphous inorganic materials). The inventors have determined that if this cooling temperature were not controlled, the physicochemical properties of the polymeric particles would be altered, in such a way that above the Tg the purified polymeric particles exhibit decreased density, hardness, and/or rigidity, which conditions the subsequent processing of the material for its disintegration and more, if they are parenteral or implantable systems. Preferably, the cooling step is carried out with sterile compressed air (inert gas) through a 0.22 μm pore size HEPA filter or with sterile inert gas at a temperature lower than that of step b) until the polymer reaches a temperature of at least 4.5 to 5.5° C. below the glass transition temperature (Tg).

In some embodiments, the comminuting step provides purified polymeric particles several millimeters in diameter. The process optionally further comprises the step of drying the purified polymeric particles by applying vacuum a temperature range about 9-35.5° C. or about 11-34.5° C., for an estimated time of about 16-32 hours, to obtain dried purified polymeric particles having a particle diameter ≥1 mm.

In some embodiments, the filter has a pore size of about 100-150 μm. Preferably, the filter is made of a pharmaceutical grade metal such as 304 stainless steel and 316 stainless steel. In some embodiments, the filtering is conducted with more than one filter in series, in parallel, in cascade, or in any combination thereof. In some embodiments, the process comprises providing a drive pump to promote or facilitate the extruding and filtering.

According to another embodiment, the extruding is conducted in a reactor equipped with agitator, e.g. blade, vane, turbine, propeller, anchor, spiral, or worm screw. Preferably, the reactor is pressurized using an inert gas selected from the group consisting of nitrogen, argon, helium, and compressed gas. Preferably, for the extruding step, once the preformed polymeric particles have partially melted in the reactor, agitation is initiated, and stirring continues until the reactor has reached a temperature of at least 4.5-5.5° C. above the melting temperature (Tm) of the polymer. During extrusion, the temperature is maintained, and agitation is stopped, the vessel is pressurized with inert gas, and a discharge valve is opened so that the molten polymer flows from the reactor (vessel) and is then filtered.

According to another embodiment, the process of the present invention, after the comminuting step, optionally further comprises an additional drying step, at least one grinding step, and/or at least one sieving step, whereby purified polymeric particles of diameter≤1 mm, e.g. microparticles with an average diameter in the range of about 100-150 μm, are obtained. Said steps can be carried out by applying vacuum for a time of at least 10 hours at room temperature in sterile conditions.

According to another embodiment, the comminuting step is conducted with a blade system.

According to another embodiment, a method as described here may comprise an additional step of sterilizing, performed at any time after the comminuting step. In a preferred embodiment, the polymeric particles are sterilized by being subjected to a dose of Beta radiation equal to or greater than 25 kGy.

According to another embodiment, the process of the present invention is carried out in a sterile environment. In some embodiments, all equipment is sterilized with nebulized or vaporized hydrogen peroxide or mixture of hydrogen peroxide with peracetic acid before polymer is placed in the reactor.

Another aspect of the invention provides a solvent-free process for removing extrinsic particles from preformed powdered biodegradable thermoplastic polymer, the process comprising the steps of a) providing powdered biodegradable thermoplastic polymer comprising said extrinsic particles; b) heating said powdered polymer to a temperature that is not more than about 5.5° C. above its melting temperature (Tm) to form molten polymer; c) filtering the molten polymer through at least one filter having a pore size of about 5-300 μm; d) cooling the extruded polymer by means of a sterile gas stream to a temperature at least 4.5-5.5° C. below the glass transition temperature (Tg) of the biodegradable thermoplastic polymer; and e) comminuting the cooled polymer to form purified powdered biodegradable thermoplastic polymer free from visible extrinsic particles of a size greater than greater than 150 μm. In some embodiments, the purified polymer exhibits substantially the same physicochemical properties and/or rheological properties as the starting preformed polymer; however, the particle size distribution of the purified polymer may or may not be different than or the same as the particle size distribution of the starting preformed polymer. In some embodiments, the purified particles are sterile.

The invention also provides a purified powdered biodegradable thermoplastic polymer free from visible extrinsic particles of a size greater than greater than 150 μm, wherein the preformed particles of said polymer have undergone solvent-free melt extrusion/filtration through at least one filter having a pore size of about 5-300 μm to form filtered polymer, wherein the melt extrusion/filtration is conducted at a temperature that is not more than about 5.5° C. above the melting temperature (Tm), and wherein the filtered polymer has been cooled with an inert gas to a temperature of at least 4.5-5.5° C. below the glass transition temperature (Tg) of the polymer. In some embodiments, the starting preformed polymer has not been dissolved in solvent during the purification step. In some embodiments the purified powdered biodegradable thermoplastic polymer has been sterilized by exposure to beta radiation. In some embodiments, the purified powdered biodegradable thermoplastic polymer is comminuted to an average particle size in the micron range and/or the nanometer range.

In some embodiments, the particles comprise PLGA or PLA. Preferably, particles comprise PLGA or PLA and have the following particle size distribution: D10: in the range of 25-55 μm, D50: in the range of 120-170 μm; and D90: in the range of 300-375 μm.

The present invention also provides biodegradable thermoplastic polymer particles characterized by having a bulk density of 0.10 to 9.0 g/cm³, a compacted density of 0.13 to 12.0 g/cm³, a residual solvent quantity of not more than 0.00%, in particular not more than 0.000%, a pyrogenic load below 1 EU/mg, a microbial load below 300 U.F.C/mg, and a particle size distribution of D10 in the range of 25-55 μm, D50 in the range of 120-170 μm, and D90 in the range of 300-375 μm, and being free of extrinsic particles greater than 150 μm in size.

The invention also provides a pharmaceutical composition comprising the sterile purified powdered biodegradable thermoplastic polymer (free from visible extrinsic particles of a size greater than greater than 150 μm), at least one drug, and at least one pharmaceutically acceptable excipient. The pharmaceutical composition may be suitable for human or animal use. The pharmaceutical composition may be a dosage form or may be suitable for making or being included in a dosage form. In some embodiments, the dosage form has been prepared using a molten form or solvent-dissolved form of the purified powdered biodegradable thermoplastic polymer. Some embodiments provide a pharmaceutical kit comprising at least one sterile purified powdered biodegradable thermoplastic polymer of the invention and at least one solvent for the at least one polymer, wherein the kit optionally further comprises at least one drug. In some embodiments, the kit is used to form an injectable depot composition that forms an extended-release implant or extended-release microparticles after administration to a subject.

In some embodiments, the invention provides a medical device comprising the sterile purified powdered biodegradable thermoplastic polymer. The medical device may or may not be implantable. The medical device optionally further comprises at least on pharmaceutical excipient and/or at least one drug. In some embodiments, the medical device is selected from the group consisting of artificial joint, cochlear implant, intraocular lens, pacemaker, cardiac implant, intrauterine contraceptive device, stent, suture, drug-eluting stent, cardiac catheter, scaffold, and urinary catheter. Other exemplary biodegradable medical devices include, but are not limited to, orthopedic pins, orthopedic screws, orthopedic plates, replacement joints, bone prostheses, cements, intraosseous devices, drug-supply devices, neuromuscular sensors and stimulators, replacement tendons, subperiosteal implants, ligation clips, electrodes, artificial arteriovenous fistulae, heart valves, vascular grafts, internal drug-delivery catheters, ventricular-assist devices, laparoscopes, arthroscopes, draining systems, dental cements, dental filling materials, skin staples, intravascular catheters, ulcer tissue dressing, burn tissue dressing, granulation tissue dressing, intraintestinal devices, endotracheal tubes, bronchoscopes, dental prostheses, orthodontic devices, intrauterine devices, and healing devices.

The invention also provides a method of treating a disease, disorder or condition, the method comprising administering to a subject in need thereof one or more doses of the pharmaceutical composition. The subject may be mammalian or non-mammalian. Preferred subjects include humans and animals.

### BRIEF DESCRIPTION OF THE FIGURES

FIG. 1 depicts a diagram that represents the solidification and cooling process of non-crystalline and partially crystalline thermoplastic polymers with specific volume change with temperature. FIG. 1 shows that there is a decrease in the specific volume with the decrease in temperature that presents a change of slope at a characteristic temperature of the material that is called the glass transition temperature, Tg, above which the polymer exhibits a viscous behavior (rubbery, elastic) and below which the polymer exhibits a brittle glass behavior.

FIG. 2 depicts a photograph taken with a dry binocular microscope, of a sample of Lactel® particles (Durect® PLGA) as received from the supplier for the beginning of the procedure object of the present invention. This image shows the presence of cellulose fibers in all polymer particles.

FIG. 3 depicts a photograph taken with a binocular microscope, of a sample of Lactel® particles (Durect's® PLGA) subjected to dissolution in an acetone solution. This image shows the presence of fibers or extrinsic particles in the solution.

FIG. 4 depicts an IR spectrophotometric image of the nature of the extrinsic particles obtained after the solution of Durect's® PLGA that were in suspension. It was determined that the extrinsic particles are mostly cellulose and polyester.

FIG. 5 depicts particles obtained after the process of the present invention with a degree of purity of at least 95% with respect to the starting polymeric particles. As can be clearly seen, there is no extrinsic particle.

FIG. 6 depicts a photograph of a solution of the particles, in acetone, obtained after the process of the present invention with a degree of purity of at least 95% with respect to the polymeric particles of departure. As can clearly be seen, no extrinsic particle is observed.

FIG. 7 depicts a photograph of sample microparticles obtained after carrying out the procedure of example 3. This image shows the total absence of extrinsic particles, presenting a degree of purity of at least 95% with respect to the starting polymeric particles. As can be clearly seen there is no extrinsic particle, and the sample of microparticles is totally homogeneous.

### DETAILED DESCRIPTION OF THE INVENTION

As used in this description, it should be understood that, unless otherwise specified, the following terms have the following meanings:

“Biodegradable” refers to a material that can be degraded or metabolized within the body, so that it is generally not removed intact.

“Biomaterial” includes all materials suitable for contact with body tissues for specific therapeutic, diagnostic, or preventive purposes. These materials must be biocompatible.

“Biocompatible” means a material that does not cause any significant adverse response from the physiological environment following interaction with tissues and body fluids and must sometimes biodegrade into non-toxic components, either chemically or physically, or a combination of both.

“Particles” according to the present invention, refers to particulate systems of various shapes with a particle size ≥1 mm. For the purpose of the present invention, the term “particle” includes any amorphous or defined particle of polymeric origin, such as granules, pellets, agglomerates, aggregates, among others. The particles are used as a final product, or as intermediate products of the manufacturing process.

“Extrinsic particles” according to the present invention, means visible particulate systems of a different nature than the starting polymeric materials, which are detectable when such polymeric materials are subjected to dissolution in a medium in which they are completely solubilized. That is, those particulate systems insoluble in a medium in which the starting polymeric material is totally soluble. This definition shall include any amorphous or defined particle of polymeric origin, such as fibers, granules, pellets, agglomerates, aggregates, among others. They may be organic or inorganic in composition.

“Granules” means formulations consisting of agglomerates of particles or powders of small size, which may be spherical or irregular in shape.

“Pellet” means a material that is compacted in the form of small spheres or cylinders by processes such as compaction, extrusion and/or spheronization (melting of the solid mass, wetting of the dry mass, extrusion of the wet or melted mass and rotation of the extruded by spheronization and subsequent drying).

“Microparticles” are spherical or non-spherical particles, with average diameters between 100 and 150 μm, preferably an average diameter of 125 μm. This group includes microcapsules, which are defined as vesicular systems in which the drug is confined in a cavity surrounded by a single membrane (usually polymeric); and microspheres, which are matrix systems in the form of spherical particles between one and several tens of microns, without a distinction between shell and core, in which the drug is dissolved or dispersed within a matrix consisting of the support materials, usually biocompatible polymers and with a large spectrum of release rates and degradative properties.μ

“Nanoparticles” are submicron particulate systems (<1 μm). According to the process used to prepare nanoparticles, nanocapsules or nanospheres can be obtained, these being morphological equivalents of microcapsules and microspheres, respectively.

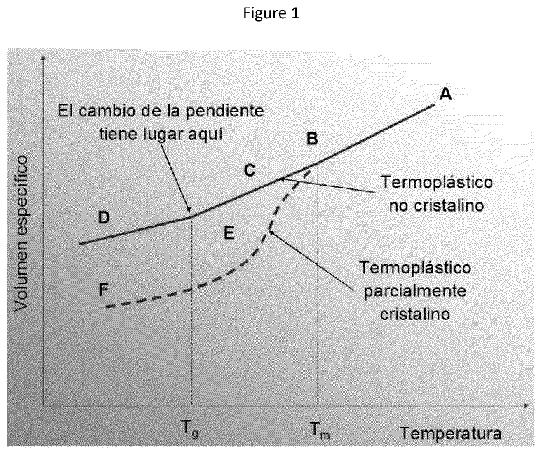

“Tg” or “glass transition temperature” means the temperature at which a thermodynamic pseudotransition occurs in glassy materials. As can be seen in FIG. 1, it is an intermediate point of temperature between the molten state (Tm) and the rigid state of the material. Tg control is important, because when thermoplastics solidify from the liquid state, they can form a non-crystalline solid or a crystalline solid. Regarding the solidification and slow cooling of non-crystalline or semi-crystalline thermoplastics, there is a decrease in the specific volume with the decrease in temperature that presents a change of slope at a temperature characteristic of the material that is called the glass transition temperature, Tg, above which the polymer presents a viscous behavior (rubbery, elastic), and underneath a brittle glass behavior. This rapid decrease is due to the packing of the polymer chains in the crystalline regions of the material, since the structure of the material is composed of crystalline regions immersed in an amorphous matrix of sub-cooled liquid, which below Tg passes to the vitreous state, leaving the structure formed by crystalline regions immersed in an amorphous vitreous matrix.

“Tm” means the melting temperature (Tm), which is the temperature at which the phase transition from solid to liquid or molten at normal atmospheric pressure occurs.

Exemplary melting temperature and glass transition temperature of some polymers are set forth below.

| | | Glass- | | Degradation |
|---|---|---|---|---|
| | Melting | Transition | Modulus | Time |
| Polymer | Point (° C.) | Temp (° C.) | (Gpa)^{a} | (months)^{b} |
| PGA | 225-230 | 35-40 | 7.0 | 6 to 12 |
| LPLA | 173-178 | 60-65 | 2.7 | >24 |
| DLPLA | Amorphous | 55-60 | 1.9 | 12 to 16 |
| PCL | 58-63 | (−65)-(−60) | 0.4 | >24 |
| PDO | N/A | (−10)-0 | 1.5 | 6 to 12 |
| PGA-TMC | N/A | N/A | 2.4 | 6 to 12 |
| 85/15 DLPLG | Amorphous | 50-55 | 2.0 | 5 to 6 |
| 75/25 DLPLG | Amorphous | 50-55 | 2.0 | 4 to 5 |
| 65/35 DLPLG | Amorphous | 45-50 | 2.0 | 3 to 4 |
| 50/50 DLPLG | Amorphous | 45-50 | 2.0 | 1 to 2 |

^{a}Tensile or flexural modulus.
^{b}Time to complete mass loss. Rate also depends on part geometry.

By purification “dry” (or dry purification or solvent-free purification) is meant a process of purification of polymeric particles in which neither water nor organic solvents is used in any of the steps of the process of the invention. This aspect is essential of the present invention because it allows for the preparation of purified particles with essentially the same structural (physicochemical) properties as those of the starting particles.

“Sterile conditions” is intended to mean environmental, equipment, installation, room and work conditions that are suitable for obtaining a non-pyrogenic product with a low microbial load; “non-pyrogenic” means a product with a pyrogenic load below 1 EU/mg (nontoxic units per milligram), and “low microbial load” means a product with less than 300 U.F.C/mg (colony-forming units per mg).

The process of the present invention is suitable for use with thermoplastic polymers (both homopolymers and copolymers) for medical use, non-parenteral, or parenteral use. Exemplary thermoplastic polymers include lactic acid polymers (L,D,DL), glycolic polymers, ε-caprolactone such as poly polymer (L-lactic), poly (D-lactic), poly (DL-lactic), polyglycolic, poly (ε-caprolactone), copolymers of L-lactic/S-lactic, L-lactic/DL-lactic acids, L-lactic/DL-lactic, L-lactic/glycolic, DL-lactic/glycolic or L-lactic/ε-caprolactone, PC (polycarbonate), PE (polyethylene), PEEK (polyether ketone), PEI (polyetherimide), PES (polyethersulfone), POM (polyoxymethylene), PP (polypropylene), PPS (polyphenylene sulfide), PPSU (polyphenylsulfone), PS (polystyrene), PSU (polysulfone), PTFE (polytetrafluoroethylene) and UHMWPE (ultra-high molecular weight polyethylene), and other known thermoplastic polymers.

The purified polymeric particles and/or microparticles of biocompatible biodegradable polymers made according to the invention exhibit a purity level of at least 95%, preferably at least 97% and more preferably 99%, without fibers or without particles of a nature different from that of the polymer of choice.

Preferably, the polymers, of which the particles to be purified are made, are selected from the group consisting of homopolymer particles and copolymers of lactic acid (L, D, DL), glycolic and/or ε-caprolactone such as poly polymer (L-lactic), poly (D-lactic), poly (DL-lactic), polyglycolic, poly (ε-caprolactone), copolymers of L-lactic/S-lactic acids, L-lactic/DL-lactic, L-lactic/glycolic, DL-lactic/glycolic DL, DL-lactic/glycolic or L-lactic/ε-caprolactone, PC, PE, PEEK, PEI, PES, POM, PP, PPS, PPSU, PS, PSU, PTFE, UHMWPE, and any combination thereof.

Although not limited to any particular bulk density, the bulk density (Pb) of polymeric particles obtained by the process of the present invention may be, for example, in the range of 0.10 g/cm³ to 9.0 g/cm³, and the compacted density (Pt) of the same can be, for example, in the range of 0.13 to 12.0 g/cm³.

The bulk density (Pb) of polymeric particles obtained by the process of the present invention and the compacted density (pt, of “tap or tapped density”) can be determined by methods and using apparatus known in the prior art. For example, as described in document WO2015/028060. In particular, Method 1 indicated by the United States Pharmacopeial Convention (USP<616>) or Method 1 indicated by European Pharmacopoeia (Ph. Eur. 7.0/2010:2.9. 3 (4).

Residual solvents are determined by methods known in the prior art, for example, according to ICH Q3C (R6) on impurities: Guideline for Residual Solvents of the European Medicines Agency (EMA/CHMP/ICH/82260/2006), published on Aug. 9, 2019. In particular, the residual quantity of specific solvents is below the limits indicated in said guide.

The absence of visible extrinsic particles can be easily determined by observing a sample of the particles under a microscope, for example, using a binocular microscope, or using infrared (IR) spectrophotometry, which allows to determine the presence of components of a chemistry different from that of biodegradable polymeric particles.

The polymeric particles obtained can be typically sterile, also referred to as pyrogen-free and/or having low microbial load; “non-pyrogenic” is understood as a product that has a pyrogenic load below 1 EU/mg (endotoxic units per milligram) and by “low microbial load” the product having less than 300 u.f.c/mg (colony forming units per milligram), determined by methods known in the art, in particular according to the United States Pharmacopeial Convention respectively by USP <85> Bacterial Endotoxins Test and USP <61> Microbial Enumeration Tests.

In specific embodiments, the polymer particles of the invention can have a microbial load of less than 100 u.f.c/g, and a pyrogen load of less than 0.05 EU/mg. These charges can be obtained, for example, after sterilizing the polymer by Beta radiation with a dose equal to or greater than 25 kGy. In addition, the process described herein may comprise an additional sterilization step performed after step any comminuting step, whereby the polymeric particles are sterilized with a dose of Beta radiation equal to or greater than 25 kGy.

### EXAMPLES

The following specific examples provided herein serve to illustrate the nature of the present invention. These examples are included for illustrative purposes only and are not to be construed as limitations on the invention claimed herein.

Thermoplastic polymers such as PLGA (lactic or glycolic acid) and PLA (polylactic acid) have been used in these examples.

The determination of bulk density (Pb) and compacted density (Pt) has been carried out by method 1 indicated by the United States Pharmacopeial Convention (USP<616>) or method 1 indicated by European Pharmacopoeia (Ph. Eur. 7.0/2010:2.9.34) using the SOTAX TD1 equipment.

The determination of pyrogenic load (EU/mg, endotoxic units per milligram) and microbial load (u.f.c./mg, colony forming units per milligram) has been carried out in accordance with the United States Pharmacopeial Convention respectively by USP <85> Bacterial Endotoxins Test and USP <61> Microbial Enumeration Tests.

### Example 1. Purification Procedure of the Thermoplastic Polymer PLGA

In this example, the aim is to purify polymeric particles of PLGA 50/50 of the Durect® brand (in particular Lactel DL-PLG (B6010-1): ester termination, intrinsic viscosity (IV) of 0.26-0.54 dL/g and apparent and compacted densities of the unpurified polymer respectively of 0.64 and 0.84 g/cm³).

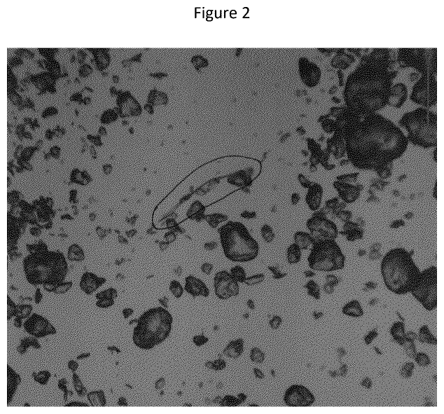

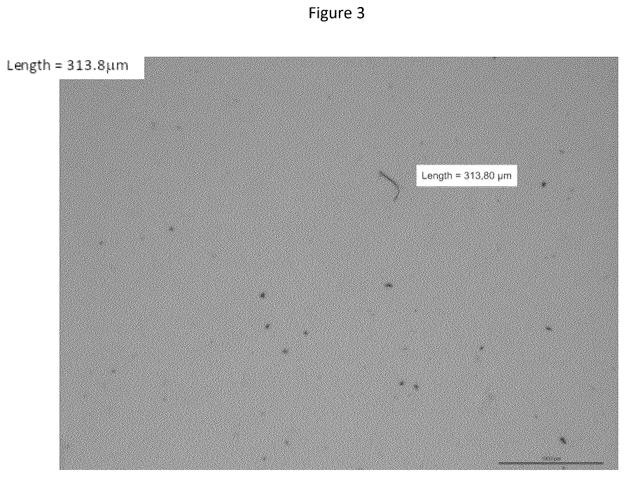

Before starting, a sample of the commercial polymer particles to be purified is analyzed under the microscope, in order to assess the degree of visible particles outside the polymer (extrinsic) to be eliminated. FIGS. 2 and 3 depicts images taken with a binocular microscope where fibers larger than 100 μm with a morphology very different from the PLGA particles that are intended to be purified can be seen. The images clear demonstrate that purchased PLGA particles (pharmaceutical grade) are not really suitable for parenterally administration and require an additional purification process.

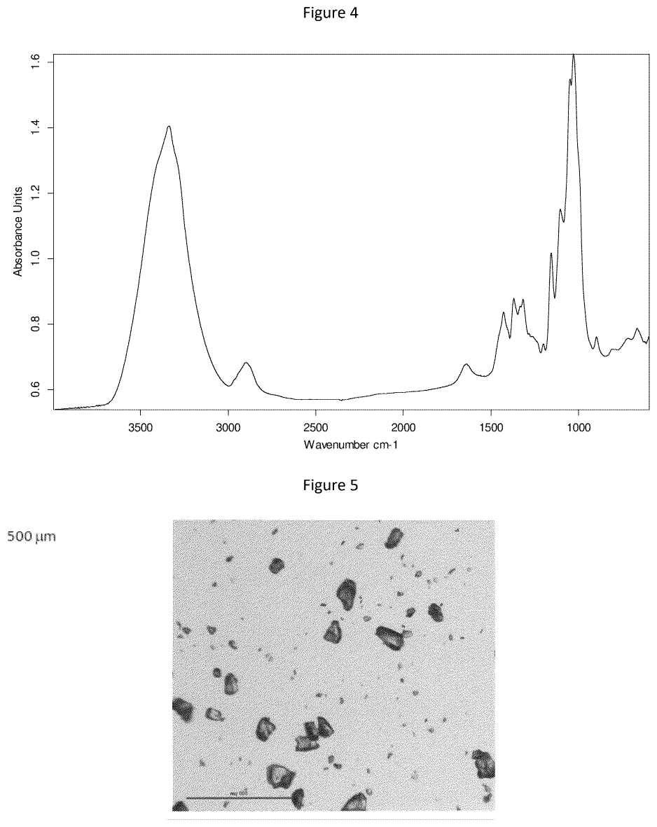

The morphologically different extrinsic particles were analyzed by FTIR spectroscopy. It was found that the extrinsic particles were for the most part cellulose fibers (FIG. 4). The particles were then subjected to the dry purification process of the invention.

The purification process begins with an extrusion stage that takes place inside a reactor. Before starting with the purification process, all equipment and materials to be used are clean and sterile. To do this, first, either proceed to perform a sterilization of all the equipment with nebulized or vaporized hydrogen peroxide or mixture of hydrogen peroxide with peracetic acid or proceed with a disinfection of all equipment with disinfectants known in the state of the art. Additionally, in the case of injectable pharmaceutical grade products, all rooms and equipment associated with the process should be sterile.

Thus, unpurified polymer particles are placed into the reactor, which is then closed and pressurized with an inert gas such as nitrogen. Next, the polymer is heated using a gradual temperature gradient of about 2° C./min±10% from room temperature. When the particles have partially melted, the agitation in the reactor is started with blades, and the agitation is continued until a temperature above the melting temperature (Tm) of the polymer is reached, that is, a temperature in a range of 20-70° C.±10%. Once these conditions are reached, the agitation is stopped, and the reactor is depressurized while maintaining the temperature. The reactor is then pressurized with nitrogen. After this, the discharge valve is opened so that the molten PLGA flows from the reactor through a filter with an average pore size of about 100 μm. By way of this filtration, contaminant (extrinsic) particles that have not been melted in the polymer mass are removed. The polymer mass is then cooled by way of a sterile inert gas stream to a temperature of at least 4.5 to 5.5° C. below Tg. The cooled polymer is then comminuted to provide polymeric particles with a particle diameter ≥1 mm.

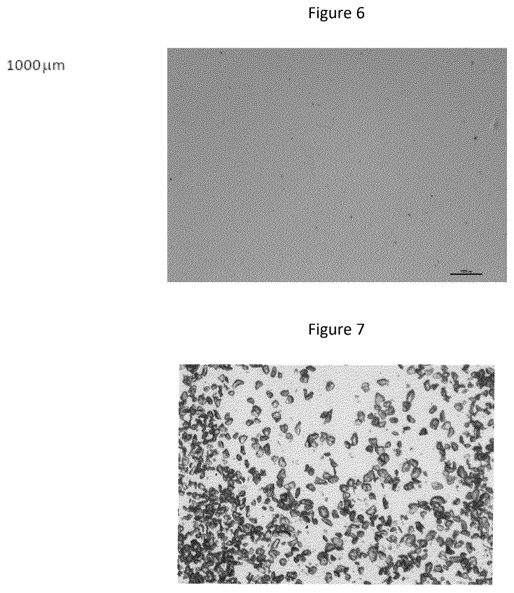

Finally, a sample of the purified particles is and analyzed with the microscope. The results indicated the cellulose fibers are no longer present in the initial commercial sample (FIGS. 5 and 6).

The purified PLGA has a specific density identical to that of the unpurified polymer. In particular, the purified PLGA has bulk and compacted densities identical to those of the unpurified starting polymer. The bulk density of the particles obtained is 0.64 g/cm³ and the compacted density is 0.84 g/cm³, both the same as those of the starting polymeric product.

In addition, due to the absence of solvent use in the purification process, the particles obtained do not contain a residual amount of solvent. The amount of residual solvents is below the detection limits, and the total amount of residual solvents is 0.000%, since it is based on a polymer not previously purified with solvents and is therefore below 0.1% the most restrictive limit for class 3 solvents of the guide: ICH Q3C (R6) on impurities: Guideline for Residual Solvents of the European Medicines Agency (EMA/CHMP/ICH/82260/2006), published on Aug. 9, 2019.

The pyrogenic load of the particles obtained is below 1 EU/mg, and the microbial load of the particles obtained is below 300 u.f.c/mg. After sterilizing the particles obtained by Beta radiation equal to or greater than 25 kGy, the microbial load of the particles obtained is below 100 CFU/g, and the pyrogen count is below 0.05 EU/mg.

### Example 2. Thermoplastic Polymer Particle Purification Procedure PLA

In this example, the aim is to purify polymeric PLA particles of the Resomer® brand. Before starting, a sample of the commercial polymer particles to be purified is analyzed under the microscope, in order to assess the content of visible extrinsic particles to be removed. Fibers (extrinsic particles) larger than 100 μm with a morphology very different from the PLA particles that are intended to be purified were observed. The image showed a clear indication that the purchased PLA particles (pharmaceutical grade) are not really suitable for parenteral administrations, so they require an additional purification process.

The extrinsic particles were analyzed by FTIR spectroscopy, where they were found identified as cellulose fibers. The particles were then subjected to the dry purification process of the invention, similar to that of Example 1.

To begin the purification process, the polymer particles are added to the reactor, which is then closed and pressurized with an inert gas such as nitrogen. Next, the polymer is heated using a gradual temperature gradient. Once the PLA particles have partially melted, the particles are agitated with a propeller until a temperature of at least 5° C.±10% above the melting temperature (Tm) of the polymer is reached. The Tm in each case is determined according to the nature and composition of the polymer, and the Tm in general terms is typically between 50° C. and 300° C., and more particularly between 50° C. and 180° C. Once these conditions are reached, the agitation is stopped, and the reactor is depressurized. The reactor is then pressurized with nitrogen. After this, the discharge valve is opened so that the molten PLA flows from the reactor through a filter with an average pore size of 100 μm. By wat of this filtration, contaminant particles that have not been melted in the polymer mass are eliminated, thereby provide a purified PLA with a specific density identical to that of the unpurified polymer.

The polymer mass is then cooled with a sterile air stream to a temperature of at least 4.5 to 5.5° C. below Tg. The cooled polymer is then comminuted to reach a particle diameter ≥1 mm.

Finally, a sample of the comminuted particles is taken after the purification process and observed again under the microscope. The results demonstrate that the cellulose fibers from the initial commercial sample, as shown in example 1, are no longer present.

### Example 3. Purification Procedure for Polymeric Microparticles

For this example, a sample of particles obtained after the procedure of examples 1 and 2 is taken and then the following steps are carried out. The particles are vacuum dried for at least 10 hours at room temperature. The particles are then ground or sieved to form microparticles having an average diameter between 100 and 150 μm. The sieving or grinding (micronization) is carried out by means of a system of in-line blades that provides dry powder with an optimal dispersion of particle sizes, e.g. D10: about 25-55 μm; D50: about 120-170 μm; D90: about 300-375 μm.

A sample of the micronized particles is analyzed under the microscope. The results (FIG. 7) demonstrate absence of the cellulose fibers observed in initial commercial sample.

### Comparative Examples

### Comparison of Density of Products Purified by Prior Art Methods and Unpurified Products:

This example compares the bulk and compacted density of a solvent-purified commercial polymer (45 RESOMER RG 503 H GMP and 26 RESOMER RG 504 H GMP), and another commercial polymer with identical unpurified characteristics (RESOMER®®® Select 5050 DLG SE-Mill).

| | | Intrinsic | | |
|---|---|---|---|---|
| | | Viscosity | | |
| | | (IV) | | |
| Polymer | Purification | (dL/g) | ρ_{b} (g/cm³) | ρₜ (g/cm³) |
| RESOMER ® | Purified per | 0.45-0.60 | 0.64 | 0.84 |
| Select 5050 | the invention | | | |
| DLG SE-Mill | | | | |
| 45 RESOMER ® | Purified with | 0.33-0.44 | 0.09 ± 0.02 | 0.11 ± 0.02 |
| RG 503 H GMP | solvents | | | |
| 26 RESOMER ® | Purified with | 0.45-0.60 | 0.09 ± 0.02 | 0.10 ± 0.01 |
| RG 504 H GMP | solvents | | | |

As shown in the table, purification using solvents results in a very low bulk and compacted density compared to the same unpurified product.

On the other hand, a polymer of similar characteristics purified with the method of the present invention results in a polymer with the same density characteristics as the starting product (Example 1).

Residual Amount of Solvent from State-of-the-Art Products, Purified by Solvent-Using Methods:

| Polymer | | Residual Solvents |
|---|---|---|
| (Sigma-Aldrich) | Purification | (GLC-HS) |
| Resomer ® | Purified with solvents | 23 PPM TOLUENE |
| RG 502 H | | 0.01% ACETONE |
| | | 0.02% TOTAL |

As can be seen, the residual amount of solvents is much higher than that of polymeric particles obtained by the process of the invention (0.00% in Example 1).

1. A solvent-free process for removing extrinsic particles from preformed powdered biodegradable thermoplastic polymer, the process comprising the steps of a) providing powdered biodegradable thermoplastic polymer comprising said extrinsic particles; b) heating said powdered polymer to a temperature that is not more than about 5.5° C. above its melting temperature (Tm) to form molten polymer; c) filtering the molten polymer through at least one filter having a pore size of about 5-300 μm to form an extruded polymer; d) cooling the extruded polymer by means of a sterile gas stream to a temperature at least 4.5-5.5° C. below the glass transition temperature (Tg) of the biodegradable thermoplastic polymer; and e) comminuting the cooled polymer to form purified powdered biodegradable thermoplastic polymer free from visible extrinsic particles of a size greater than greater than 150 μm.

2. The process of claim 1, wherein a) the preformed polymer has not been dissolved in solvent during the purification step; b) the purified powdered biodegradable thermoplastic polymer has been sterilized by exposure to beta radiation; c) the purified powdered biodegradable thermoplastic polymer has been comminuted to an average particle size in the millimeter range, micron range, or nanometer range; and/or d) the preformed polymer has not been purified or previously, or has not been previously purified with a method using solvents.

3. The process of claim 1, wherein the filtering is performed at positive pressure greater than 0.1 bar in the presence of an inert gas.

4. The process of claim 2, wherein the filtering is performed with a plurality of filters located in series, in parallel, in cascade or in any of its combinations.

5. The process of claim 1 further comprising the step of drying the purified powdered biodegradable thermoplastic polymer by applying vacuum for a period of at least 10 hours at room temperature in sterile conditions where particles of diameter ≤1 mm are obtained.

6. The process of claim 1 further comprising the step of sterilizing purified the powdered biodegradable thermoplastic polymer by subjecting it to a dose of beta radiation equal to or greater than 25 kGy.

7. The process of claim 1, wherein said comminuting is conducted with a system of blades in line.

8. The process of claim 1, wherein step b) is carried out in a reactor with agitation by blade or vane, turbine, propeller, anchor, spiral or screw without end.

9. The process of claim 1, wherein the process is conducted in the presence of an inert gas selected from the group consisting of nitrogen, argon, helium and compressed gas.

10. The process of claim 1, wherein the biodegradable thermoplastic polymer is a homopolymer or copolymer selected from the group consisting of lactic acid (L, D, DL), glycolic, and ε-caprolactone.

11. The process of claim 11, wherein the biodegradable thermoplastic polymer is selected from the group consisting of poly (L-lactic), poly (D-lactic), poly (DL-lactic), polyglycolic, poly (ε-caprolactone), copolymer of L-lactic/S-lactic acids, copolymer of L-lactic/DL-lactic, copolymer of L-lactic/glycolic, copolymer of DL-lactic/glycolic, copolymer of L-lactic/ε-caprolactone, PLGA, PLA, PC, PE, PEEK, PEI, PES, POM, PP, PPS, PPSU, PS, PSU, PTFE and UHMWPE.

12. The process of claim 11, wherein the purified powdered biodegradable thermoplastic polymer a) exhibits the following size distribution: D10: between 25-55 μm; D50: between 120-170 μm; and D90: between 300-375 μm; b) exhibits a bulk density of 0.10 to 9.0 g/cm 3; c) exhibits a compacted density of 0.13 to 12.0 g/cm³d d) has a residual solvent content of 0.00%; e) exhibits a pyrogenic load below 1 EU/mg and a Microbial load below 300 U.F.C/mg; and/or f) has a polymer purity level of at least 95%.

13. A purified powdered biodegradable thermoplastic polymer made according to the process of claim 1.

14. A pharmaceutical composition comprising at least one drug, at least one pharmaceutically acceptable excipient, and the purified polymer of claim 13.

15. A dosage form comprising the pharmaceutical composition of claim 14.

16. A medical device comprising the purified polymer of claim 13.

17. A method of treating a disease, disorder or condition, the method comprising administering to a subject in need thereof one or more doses of the pharmaceutical composition of claim 14.

18. A method of treating a disease, disorder or condition, the method comprising administering to a subject in need thereof one or more dosage forms according to claim 15.

19. A method of treating a disease, disorder or condition, the method comprising administering to or implanting in a subject in need thereof one or more medical devices according to claim 16.
